(19) United States
(12) Patent Application Publication
(10) Pub. No.: US 2007/0238742 A1
(43) Pub. Date: Oct. 11, 2007

(54) METHODS OF PREPARATION AND RESOLUTION OF E/Z ISOMERS OF VINYLFURO[2,3-D]PYRIMIDINE AND THEIR BIOLOGICAL ACTIVITIES AND RELATED COMPOSITIONS AND METHODS OF TREATMENT

(76) Inventors: Aleem Gangjee, Allison Park, PA (US); George R. Martin, Rockville, MD (US); Kumar Gadamasetti, Belmont, CA (US)

Correspondence Address: ECKERT SEAMANS CHERIN & MELLOTT, 600 GRANT STREET, 44TH FLOOR, PITTSBURGH, PA, 15219, US

(21) Appl. No.: 11/734,831

(22) Filed: Apr. 13, 2007

Related U.S. Application Data
(63) Continuation-in-part of application No. 11/687,888, filed on Mar. 19, 2007.
(60) Provisional application No. 60/784,128, filed on Mar. 20, 2006.

(51) Int. Cl. A61K 31/519; C07D 491/02

(52) U.S. Cl. 514/260.1; 544/278

(57) ABSTRACT

Stereoselective methods for preparing the individual isomers, E- and Z-2,4-substituted-5-vinylfuro[2,3-d]pyrimidine and pharmaceutically acceptable salts, solvates, and prodrugs thereof using selective synthetic conditions are provided. This class of pyrimidine compounds function as receptor tyrosine kinase inhibitors during angiogenesis and resists the development of new blood vessels in tumors as well as inhibit the folate pathway required for cell growth. The isomers of these compounds are separated by physical, chromatographic, and/or HPLC methods. Their biological activities are described. Also provided are methods of treating diseases associated with angiogenesis in a patient comprising administering the isolated E- and Z-isomers of the composition of the present invention.

### CROSS REFERENCE TO RELATED APPLICATION

This application is a continuation-in-part of U.S. application Ser. No. 11/687,888, filed Mar. 19, 2007, which claims priority to U.S. Provisional Application No. 60/784,128, filed Mar. 20, 2006, both of which are incorporated herein by reference.

### GOVERNMENT SUPPORT

This invention was supported in part by a National Institutes of Health U.S. Department of Health and Human Services Grant under Contract No. RO1CA98850. The Government may have certain rights in the invention.

### BACKGROUND OF THE INVENTION

1. Field of the Invention

This invention relates to compositions and methods of preparation of stereospecific pyrimidine compounds and pharmaceutically acceptable salts, solvates and prodrugs thereof. Methods of treatment using these compounds also are provided. The present compounds have been found useful as antitumor and antiangiogenic agents.

2. Description of the Prior Art

Angiogenesis, the formation of new blood vessels, occurs during development and in normal adults during wound healing, pregnancy, and corpus luteum formation. Although angiogenesis is limited in normal adults, it is induced in many disease states including cancer, diabetic retinopathy, rheumatoid arthritis, psoriasis, atherosclerosis, and restenosis.

Tumors require angiogenesis to grow beyond 1-2 mm³. The increased blood flow to the tumor allows for continued growth as well as metastasis, as successful metastasis requires the presence of blood vessels to allow for the tumor cells to enter the circulation. The close interplay between angiogenesis and metastasis contributes to the poor prognosis seen in patients with highly angiogenic tumors (Chemington et al., Cancer Res., pp. 1-38, 2000).

Some of the most well characterized regulators of angiogenesis are growth factors and receptor tyrosine kinases (RTKs) involved in the migration and proliferation of endothelial cells. Of primary interest for angiogenesis are Flt-1 and Flk-1/KDR, the receptors for vascular endothelial growth factor (VEGF), as well as Tie 1 and Tie 2/Tek, the receptors for angiopoietins. These four receptors are expressed primarily on endothelial cells and play a direct role in angiogenesis. Additional RTKs with broader expression patterns implicated in angiogenesis are platelet-derived growth factor receptors (PDGFRs); fibroblast growth factor receptors (FGFRs); the hepatocyte growth factor/scatter factor (HGF/SF) receptor; Met; and epidermal growth factor receptors (EGFRs), although it is thought that EGFR likely to acts predominantly in directly driving the growth of tumor cells rather than through angiogenesis (Chemington et al., Cancer Res., pp. 1-38, 2000)

VEGF is a dimeric protein also known as vascular permeability factor because it acts on endothelial cells to regulate permeability of those cells as well as their proliferation. These two activities are mediated through its tyrosine kinase receptors, VEGFR1/Flt-1 and VEGFR2/Flk-1/KDR (KDR is the human homologue of Flk-1). VEGF and its receptors are expressed in angiogenic tissues during development, wound healing and other situations when angiogenesis occurs. The role of VEGF in tumor angiogenesis has also been clearly demonstrated using tumor models in rodents (reviewed in Hanahan, 1997; Shawver et al., 1997); and extensive literature exists linking VEGF with human cancers such as pulmonary adenocarcinoma (Takanami et al., 1997) and non-small cell carcinoma (NSCLC) (Fontanini et al., 1999; Takahama et al., 1998; Ohta et al., 1996). Survival of patients with VEGF-positive tumors was significantly less than patients with VEGF-negative tumors. For example, in one study of non-small cell carcinoma (NSCLC), patients with low VEGF levels had a median survival time of 151 months, whereas those with high VEGF expression had a mean survival time of only 8 months (Ohta et al., 1996).

VEGF and its receptors, in particular, serve as excellent targets for anti-angiogenesis therapy because KDR is an endothelial cell-specific VEGF receptor expressed primarily during the angiogenic process. The VEGF signaling cascade has been validated as a target for therapeutic intervention by several methods. (See, e.g., Saleh et al., 1996; Claffey et al., 1996; Kim et al., 1993; and Asano et al., 1995).

Epidermal growth factor (EGF) is one of several naturally occurring proteins that promotes normal cell proliferation in a tightly regulated manner by binding to its receptor, EGFR, and sending growth signals via the receptor tyrosine kinase enzyme activity to the nucleus of the cell and thus controlling growth. In many human cancers, EGFR is either overexpressed or mutated, leading to aberrant signaling and the development of a tumor; thus inhibition of EGF receptor kinase is also a target in anti-tumor therapy.

Various pyrimidine systems, such as the pyrido[2,3-d]pyrimidine ring system, have been studied due to their involvement in the inhibition of dihydrofolate reductase (DHFR) enzymes activity. Because DHFR reduces dihydrofolate to tetrahydrofolate, inhibition of DHFR deprives the cell of tetrahydrofolate, without which the cell cannot produce 5,10-methylenetetrahydrofolate. 5,10-Methylenetetrahydrofolate is essential for cell growth. The inhibition of DHFR by the compounds, and pharmaceutically acceptable salts thereof, of this invention therefore results in the inhibition of DNA synthesis and leads to cell death. Methotrexate (MTX), trimetrexate (TMQ), piritrexim (PTX) and other folic acid analogues function as inhibitors of cell growth by similar mechanisms involving the inhibition of dihydrofolate reductase.

Drugs useful for the reduction of cancerous cells also are known.

Elslager, Edward F., et al., “Folate Antagonists. 20. Synthesis and Antitumor and Antimalarial Properties of Trimetrexate and Related 6-[(Phenylamino)methyl]-2,4-quinazolinediamines;” J. Med. Chem., 26:1753-1760, 1983, discloses the preparation of quinazolinediamines. This article states that the quinazolinediamines exhibit potent antimalarial, antibacterial and antitumor activity.

Methods of synthesizing diaminopyrido[2,3-d]pyrimidines having various substituents are known. See Hurlbert, B. S., et al., “Studies on Condensed Pyrimidine Systems. XXIII. Synthesis of 2,4-Diaminopyrido[2,3-d]pyrimidines from .beta.-Keto Esters;” J. Med. Chem., 11:703-707, 1968, and Hurlbert, B. S., and Valenti, B. F., “Studies on Condensed Pyrimidine Systems. XXIV. The Condensation of 2,4,6-Triaminopyridimine with Malondialdehyde Derivatives;” J. Med. Chem., 11:708-710, 1968.

Hurlbert, B. S., et al., “Studies on Condensed Pyrimidine Systems. XXV. 2,4-Diaminopyrido[2,3-d]pyrimidines. Biological Data;” J. Med. Chem., Vol. 11, pp. 711-717 (1968), discloses the antimicrobial activities of several subgroups of pyridopyrimidines. This article states that 2,4-diaminopyrido[2,3-d]pyrimidines bearing alkyl and aralkyl substituents in the pyrimidine moiety are inhibitors of dihydrofolate reductase having antibacterial and antiprotozoal activity and that these compounds potentiate sulfonamides.

Grivsky, E. M., et al., “Synthesis and Antitumor Activity of 2,4-Diamino-6-(2,5-dimethoxybenzyl)-5-methylpyrido[2,3-d]pyridimine;” J. Med. Chem., 23:327-329, 1980, discloses the synthesis of 2,4-diamino-6-(2,5-dimethoxybenzyl)-5-methylpyrido[2,3-d]pyridimine (BW301U,7). This article states that BW301U,7 is as effective as methotrexate as an inhibitor of dihydrofolate reductase purified from human leukemic cells and, in contrast to metoprine, has minimal activity as an inhibitor of histamine metabolism.

Shih et al., “LY231514, a Pyrrolo[2,3-d]pyrimidine-based Antifolate That Inhibits Multiple Folate-requiring Enzymes;” Cancer Research, 57:1116-1123, 1997, teaches a pyrrolo[2,3-d]pyrimidine-based antifolate that inhibits multiple folate-requiring enzymes. A classical or glutamic acid substituted pyrrolo pyrimidine is disclosed.

Taylor et al., “A Dideazatetrahydrofolate Analogue Lacking a Chiral Center at C-6, N-[4-[2-(2-Amino-3,4-dihydro-4-oxo-7H-pyrrolo[2,3-d]pyrimidin-5-yl)ethyl]_{b} enzoyl]-L-glutamic Acid, Is an Inhibitor of Thymidylate Synthase;” J. Med. Chem., 35:4450-4454, 1992, also teaches a classic pyrrolo pyrimidine compound useful in the inhibition of thymidylate synthase. Taylor reports other pyrrolo pyrimidine compounds in U.S. Pat. Nos. 4,996,206; 5,028,608; 5,248,775; 5,254,687; and 5,344,932.

Werbel, Leslie, M., et al., “Synthesis and Antimalarial Activity of a Series of 2,4-Diamino-6-[(N-alkylanilino)methyl]quinazolines [1,2];” J. Heterocyclic Chem., 24:345-349, 1987, discloses the synthesis of N6 substituted quinazoline dihydrofolate reductase inhibitors. This article states that these analogs demonstrate substantial activity against Plasmodium berghei infections in mice.

Piper, J. R., et al., “Syntheses and Antifolate Activity of 5-Methyl-5-deaza Analogues of Aminopterin, Methotrexate, Folic Acid, and N.sup.10-Methylfolic Acid;” J. Med. Chem., 29:1080-1087, 1986, discloses that 5-methyl-5-deaza analogues of aminopterin and methotrexate are much more growth inhibitory than methotrexate.

Pyrido[2,3-d]and [3,2-d]pyrimidines are also disclosed in U.S. Pat. Nos. 5,346,900 and 5,508,281, and co-pending application Ser. Nos. 08/515,491 and 08/660,023 all of which are hereby expressly incorporated by reference.

Pyrrolo[2,3-d]pyrimidines are disclosed by Gangjee et al. in “Novel 2,4-diamino-5-substituted-pyrrolo[2,3-d]pyrimidines As Classical and Non-Classical Antifolate Inhibitors of Dihydrofolate Reductases;” J. Med. Chem., 38:2158-2165, Jun. 6, 1995.

Gangjee, A., et al., “Classical and Non-Classical Furo[2,3-d]Pyrimidines As Novel Antifolates Synthesis and Biological Activities;” J. Med. Chem., 37:1169-1176, 1994, discloses furo[2,3-d]pyrimidines.

Mavandadi et al., disclose 5-substituted classical and nonclassical 2,4-diaminopyrrolo[2,3-d]pyrimidines as antitoxoplasma, antipneuomocystis and antitumor agents in J. Med. Chem., 40:1173-1177, 1997. Mavandadi et al. also disclose use of pyrrolo[2,3-d]pyrimidines as nonclassical inhibitors of thymidylate synthase in J. Med. Chem., 39:4563-4568, 1996.

Racemic compounds commonly are used as therapeutic agents. However, the activity of the corresponding single isomers have been known to have diverse biological activity. Geometric isomers (E- and Z-isomers) and epimers are diastereomers as well as stereoisomers, having different spatial arrangements of atoms; consequently they are different compounds. As a result of their different configurations, their interactions with protein domains will be different. For example, the antipsychotic activity of doxepin Z-isomer has been found to be significantly greater than the corresponding E-isomer. Because thalidomide has a chiral carbon atom, it exists as two enantiomers. Tests with mice have suggested that only one enantiomer is teratogenic creating malformations in embryos, whereas the other isomer possesses therapeutic activity.

It previously has been known to provide compounds which are based on the known crystal structures of the VEGF receptor kinase and other factors, synthesized and assessed for biological activity (Gangjee, A et al., Bioorg. Med. Chem., 13:5475-91, 2005; U.S. Pat. No. 6,962,920). Design of these compounds was based on modifying a furopyrimidine scaffold expected to exhibit antifolate activity. To this scaffold, various functional groups were attached with the intent that the compounds would also bind to and inhibit the kinase domain of the VEGF receptor-2. Relevant testing established that certain of the compounds inhibited dihydrofolate reductase as well as having the ability to inhibit the VEGF and PDGF receptor kinases and inhibit angiogenesis in the chorioallantoic membrane assay. In designing these compounds, a vinyl group was used to bridge the furopyrimidine scaffold to various attached groups in order that the functional groups align appropriately in a linear fashion into the kinase domain to maximize binding. The presence of the vinyl group in the molecule creates stereoisomerism, producing mixtures of compounds expected to vary in their chemical properties and biological activities as noted with other stereoisomeric mixtures.

Thus, the preparation of single isomers of biologically active agents can be doubly beneficial in improving therapeutic responses while limiting adverse actions.

In general, it is highly desirable to develop new antiangiogenic compounds which inhibit formation of new blood vessels and development of a new blood supply, as these can selectively target various tumor types and prevent growth of circulation in the tumor and inhibit metastasis. Because angiogenesis is limited in healthy adults, compounds which inhibit angiogenesis can selectively target tumors as compared with other compounds and anti-cancer agents using other modes of action, which often indiscriminately act on tumor and healthy cells alike.

There remains a need, therefore, for compounds and methods of preparing compounds which provide the desired enzyme inhibition with a high degree of selectivity and low toxicity as well as for methods of treating diseases using such compounds.

### SUMMARY OF THE INVENTION

The present invention meets this need by providing a stereoselective method of preparing isolated E- and Z-isomers of 2,4-diamino-5-substituted vinylfuro[2,3-d]pyrimidine compounds and pharmaceutically acceptable salts, solvates and prodrugs thereof, comprising synthesizing the isolated E- and Z-isomers using at least one 2,4-substituted-5-(chloromethyl)furo[2,3-d]pyrimidine and at least one 2-substituted ketone using reaction conditions and reagents; and separating the isolated E- and Z-isomers using at least one method selected from the group consisting of physical separation, chromatography and HPLC, wherein the isolated E- and Z-isomers each have the following composition:

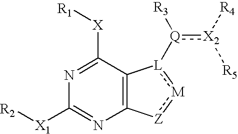

wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkenyl, a heteroaroyl and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond, and
wherein said M is a carbon when said third chemical bond is a single bond.

In an additional aspect of the present invention, a composition of 2,4-substituted-5-vinylfuro[2,3-d]pyrimidine and pharmaceutically acceptable salts, solvates, and prodrugs thereof is provided, comprising: 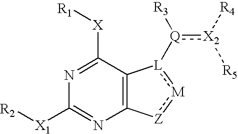
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkeyl, and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond,
wherein said M is a carbon when said third chemical bond is a single bond, and
wherein said composition contains a mixture of E- and Z-isomers that are isolatable.

In a further aspect of the present invention, a method of treating at least one disease in a patient is provided, comprising administering to the patient in a pharmaceutically effective amount either one or both of the isolated E-isomer or the isolated Z-isomer of the composition of the present invention.

In an additional aspect of the present invention, a method to reduce aberrant angiogenesis in a patient afflicted with aberrant angiogenesis is provided, comprising administering in a pharmaceutically effective amount to the patient either or both isolated E-isomer or isolated Z-isomer of the composition of the present invention.

It is an object of the present invention, therefore, to provide isolated E- and Z-isomers of 2,4-diamino-5-substituted vinylfuro[2,3-d]pyrimidine compounds and pharmaceutically acceptable salts, solvates and prodrugs thereof, having antitumor and/or anti-angiogenic activity.

It is an additional object of the present invention to provide isolated E- and Z-isomers of 2,4-diamino-5-substituted vinylfuro[2,3-d]pyrimidine compounds and pharmaceutically acceptable salts, solvates and prodrugs thereof, for substantially inhibiting receptor tyrosine kinase(s) and/or dihydrofolate reductase and/or thymidylate synthase enzymes.

It is a further object of the present invention to provide a stereoselective method for preparing isolated isolated E- and Z-isomers of 2,4-diamino-5-substituted vinylfuro[2,3-d]pyrimidine compounds and pharmaceutically acceptable salts, solvates and prodrugs thereof.

It is an additional object of the present invention to provide a method of treating various diseases such as cancer by administering isolated E- and Z-isomers of 2,4-diamino-5-substituted vinylfuro[2,3-d]pyrimidine compounds and pharmaceutically acceptable salts, solvates and prodrugs thereof.

It is a further object of the present invention to provide a method of preparing single isomers of biologically active agents which are beneficial for improving therapeutic responses while limiting adverse actions.

These and other aspects of the present invention will be more fully understood from the following detailed description of the invention and reference to the illustration appended hereto.

### BRIEF DESCRIPTION OF THE DRAWINGS

FIG. 1 is a scheme of the synthesis and isolation by column chromatography of the E-isomer (Compound 1) of the E/Z-mixture 5.

FIG. 2 is an HPLC analytical chromatogram of Compound 5 in the E/Z mixture.

FIG. 3 is a preparative chromatogram of Compound 5 in the E/Z mixture.

### DESCRIPTION OF THE PREFERRED EMBODIMENTS

The present invention provides a stereoselective method of preparing isolated E- and Z-isomers of 2,4-diamino-5-substituted vinylfuro[2,3-d]pyrimidine compounds and pharmaceutically acceptable salts, solvates and prodrugs thereof, comprising synthesizing the isolated E- and Z-isomers using at least one 2,4-substituted-5-(chloromethyl)furo[2,3-d]pyrimidine and at least one 2-substituted ketone using various reaction conditions and reagents utilizing a Witting coupling reaction; and separating the isolated E- and Z-isomers using at least one method selected from the group consisting of physical separation, chromatography and HPLC, wherein the isolated E- and Z-isomers each have the following composition:

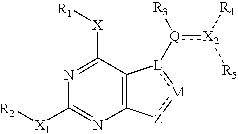

wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkenyl, a heteroaroyl and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond, and
wherein said M is a carbon when said third chemical bond is a single bond.

In the stereoselective method of preparing isolated E- and Z-isomers of the present invention, R₁ and R₂ can include the same or different substituents selected from the group of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, or a cyclic lower alkyl group with 1 to 6 backbone carbons.

Additionally, in the stereoselective method of preparing isolated E- and Z-isomers of the present invention, of the present invention, R₃, R₄ and R₅ can include the same or different substituents selected from the group consisting essentially of mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, or a cyclic lower alkyl group with 1 to 6 backbone carbons.

In the stereoselective method of preparing isolated E- and Z-isomers of the present invention, either or both of the isolated E-isomers or the isolated Z-isomers inhibit at least one tyrosine kinase during angiogenesis, and thus either or both of the isolated E-isomers or the isolated Z-isomers are anti-angiogenic agents.

Additionally, in the stereoselective method of preparing isolated E- and Z-isomers of the present invention, either or both of the isolated E-isomers or the isolated Z-isomers inhibit a folate pathway required for cell growth.

Furthermore, in the stereoselective method of preparing isolated E- and Z-isomers of the present invention, either or both of the isolated E-isomers or the isolated Z-isomers are anti-cancer agents.

In a further embodiment of the present invention, a composition of 2,4-substituted-5-vinylfuro[2,3-d]pyrimidine and pharmaceutically acceptable salts, solvates, and prodrugs thereof is provided, comprising: 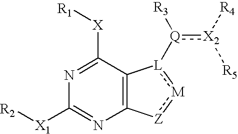
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkeyl, and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond,
wherein said M is a carbon when said third chemical bond is a single bond, and
wherein said composition contains a mixture of E- and Z-isomers that are isolatable.

In the composition of the present invention, R1 and R2 can include the same or different substituents selected from the group consisting essentially of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, or a cyclic lower alkyl group with 1 to 6 backbone carbons.

Additionally, in the composition of the present invention, R₃, R₄ and R₅ can include the same or different substituents selected from the group consisting essential of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

Furthermore, in the composition of the present invention, the E-isomers and the Z-isomers are isolated from the composition.

The isolated E-isomers and the isolated Z-isomers of the present invention each have been found to have both cytostatic and cytotoxic activity. Specifically, the isolated E-isomers and the isolated Z-isomers have been found to inhibit growth factors, such as, without limitation, VEGF and PDGF. The receptor for VEGF is involved in the initial phases of angiogenesis and the receptor for PDGF is involved in the stabilization of new capillaries.

In addition, the isolated E-isomers or the isolated Z-isomers have been shown to inhibit at least one tyrosine kinase during angiogenesis and to inhibit a dihdyrofolate reductase (DHFR) pathway required for cell proliferation.

In an additional embodiment of the present invention, a method of treating at least one disease in a patient is provided, comprising administering to the patient in a pharmaceutically effective amount either one or both of the isolated E-isomer or the isolated Z-isomer of the composition of the present invention.

In a further embodiment of the present invention, a method to reduce aberrant angiogenesis in a patient afflicted with aberrant angiogenesis is provided, comprising administering in a pharmaceutically effective amount either or both isolated E-isomer or isolated Z-isomer of the composition of the present invention to the patient.

In the methods of the present invention, both the isolated E-isomer and the isolated Z-isomer can be administered simultaneously or the E-isomer can be administered at one time point and the Z-isomer can be administered at a different time point to the patient.

Diseases that can be treated according to the methods of the present invention include, without limitation, rheumatoid arthritis, wet form of macular degeneration or cancer.

For example, it is within the scope of the present invention that the E-isomer can be administered in a pharmaceutically effective amount to the patient at one time point to treat one disease and then the Z-isomer can be administered in a pharmaceutically effective amount at a different time point to treat the same disease or to treat a different disease in the patient.

Another example within the scope of the present invention includes administering simultaneously the isolated E-isomer in a pharmaceutically effective amount and the isolated Z-isomer in a pharmaceutically effective amount to the patient to treat one or more diseases in the patient.

The E-isomer and the Z-isomer each has a unique mode of action at a specific pharmaceutically effective amount for treating one or more diseases in a patient.

Furthermore, simultaneous administration of a pharmaceutically effective amount of the E-isomer and a pharmaceutically effective amount of the Z-isomer provides a more effective treatment for one or more diseases in a patient than if a mixture of the E-isomer and the Z-isomer, as naturally found in the composition of the present invention, is administered to the patient.

The isolated E- and Z-isomers of the present invention inhibit at least one tyrosine kinase during angiogenesis as well as a folate pathway required for cell growth. Thus, the E- and Z-isomers of the present invention are anti-angiogenic and anti-cancer agents.

As used herein, the term “pharmaceutically acceptable salts and solvates” means salts or solvates of the present pyrimidine compounds suitable for use in pharmaceutical applications. One skilled in the art would easily be able to determine whether a salt or solvate form of any given compound is suitable for use as a pharmaceutical. Examples of pharmaceutically acceptable salts include, but are not limited to, acetate, formate, glucuronate, ethantate, and sulfonate. Other examples include alkaline metal, alkaline earth metal, other non-toxic metals, ammonium and substituted ammonium salts such as the sodium, potassium, lithium, calcium, magnesium, aluminum, zinc, ammonium, trimethyl ammonium, triethyl ammonium, tetrabutyl ammonium, pyridinium and substituted pyridinium salts.

As used herein, “pharmaceutically acceptable prodrugs” means any prodrug formulation of the present compounds. A prodrug will be understood by those skilled in the art as a chemical compound that is converted into an active curative agent by processes within the body. Other formulations comprising the pyrimidine compounds described herein also are within the scope of the present invention. Salts, solvates and prodrugs of the compounds of the present invention can be made by standard methods well known to those skilled in the art.

As used herein, the terms “treating” and “treatment” are used generically throughout to refer to both therapeutic and prophylactic treating/treatment that is effected by inhibition of receptor tyrosine kinases (referred to generally as “receptor tyrosine kinase”), and/or of DHFR.

As used herein, the term “disease” means various types of cancer including, but not limited to, leukemia, lung cancer, colon cancer, CNS cancer, melanoma, ovarian cancer, renal cancer, prostate cancer, breast cancer and other diseases, such as, without limitation, wet form of macular degeneration and rheumatoid arthritis

As used herein, the term “patient” means adult members of the animal kingdom, including, but not limited to, human beings.

A method of treating a patient for an illness according to the present invention comprises administering an effective amount of either the isolated E-isomer or the isolated Z-isomer or administering simultaneously effective amounts of the isolated E-isomer and the isolated Z-isomer of the compositions of the present invention.

As used herein, the term “effective amount” refers to that amount of the E-isomer or the Z-isomer required to bring about a desired effect in a patient. The desired effect will vary depending on the illness being treated. For example, the desired effect may be reducing tumor size, destroying cancerous cells, preventing metastasis or reducing symptoms associated with the various other diseases listed above and contemplated as being within the treatment methods of the present invention. On its most basic level, an effective amount is that amount needed to inhibit the receptor tyrosine kinase(s) generally and/or DHFR. Any amount of inhibition will yield a benefit to a patient and is therefore within the scope of the invention.

It will be appreciated that the effective amount will vary from patient to patient depending on such factors as the illness being treated, the severity of the illness, the size of the patient being treated, the patient's ability to mount an immune response, and the like. The determination of an effective amount for a given patient is within the skill of one practicing in the art. Typically an effective amount will be determined by evaluating potency in standard ex vivo cellular systems, followed by preclinical and clinical in vivo assessment.

Administration can be by any means known in the art, such as parenterally, orally or topically. The pyrimidine compound can be contained within a suitable pharmaceutical carrier for administration according to the present methods. “Suitable pharmaceutical carrier” refers to any pharmaceutical carrier known in the art that will solubilize the present compounds and will not give rise to compatibility problems and includes any and all solvents, dispersion media, coatings, antibacterial and antifungal agents, isotonic and absorption delaying agents, and the like. The use of such media and agents for pharmaceutical use is well known in the art. Use of any of these media or agents is contemplated by the present invention, absent compatibility problems with the compounds of the present invention. Preferred carriers include physiologic saline and 5% dextrose.

It is especially advantageous to formulate parenteral compositions in dosage unit form for ease of administration and uniformity of dosage. Dosage unit form as used herein refers to physically discrete units suited as unitary dosages for the patients being treated, each unit containing a predetermined quantity or effective amount of pyrimidine compound to produce the desired effect in association with the pharmaceutical carrier. The specification for the dosage unit forms of the present invention are dictated by and directly dependent on the particular compound and the particular effect to be achieved.

### EXAMPLES

The following examples are intended to illustrate the invention, and should not be construed as limiting the invention in any way. Standard test procedures familiar to those skilled in the art were used in the examples, such as those procedures described by Gangjee, A., et al., in “Effect of bridge region variation on antifolate and antitumor activity of classical 5-substituted 2,4-diaminofuro[2,3-d]pyrimidines,” J. Med. Chem., 38:3798-3805, 1995; and “Novel 2,4-diamino-5-substituted-pyrrolo[2,3-d]pyrimidines As Classical and Non-Classical Antifolate Inhibitors of Dihydrofolate Reductases,” J. Med. Chem., 38:2158-2165, Jun. 6, 1995, and references disclosed therein, as well as Angiogenesis Protocols, J. Clifford Murray ed., Humana Press, 2001.

### Example 1

### Synthesis and Separation of Pure E-Isomer 1

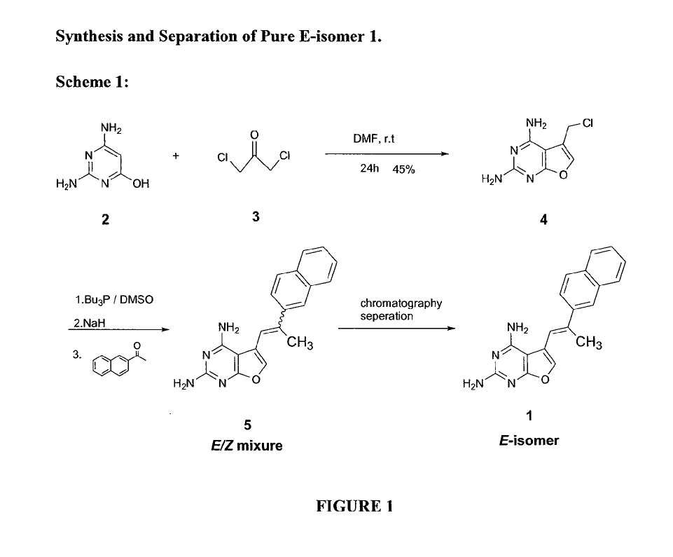

The E-isomer (Compound 1) of the E/Z-mixture 5 was synthesized and isolated by column chromatography as indicated in FIG. 1.

### Synthesis of 2,4-Diamino-5-chloro-methylfuro[2,3-d]pyrimidine (4) (Ref: Gangjee, A et al., Bioorg. Med. Chem., 13:5475-5491, 2005).

2,4-Diamino-6-hydroxy-pyrimidine 2 (6.76 g, 0.052 mol) and 1,3-dichloroacetone 3 (6.61 g, 0.051 mol) were stirred in anhydrous DMF 30 mL at room temperature under nitrogen. The resulting mixture became a clear solution (orange) after 1.5 hours and reformed to a suspension again after 2-2.5 hours. The resulting mixture was stirred overnight for 20 hours. The resulting precipitate was filtered and washed with ether followed by drying with a vacuum pump over P₂O₅ in a desiccator to obtain 8.60 g (85%) of a light yellow compound. This compound was dissolved in hot methanol, and silica gel (25 g) was added to the solution and then evaporated to dryness. The silica gel plug obtained was poured on top of a dry silica gel column (4 cm×40 cm) and eluted with 9:1 CHCl₃/CH₃OH. Fractions corresponding to the product (TLC R_{f}=0.6 CHCl₃/CH₃OH 9:1) were pooled and evaporated to dryness under reduced pressure to obtain the analytical samples 4.37 g (45%) of 4: mp: 176-182° C.; TLC R_{f}=0.6 (CHCl₃:CH₃OH=9:1). ¹H NMR (DMSO-d₆): δ 4.90 (s, 2H, 8-CH₂), 6.13 (s, 2H, 4-NH₂), 6.54 (s, 2H, 2-NH₂), 7.48 (s, 1H, 6-CH). This synthesis resulted in the production of 4.37 g of compound 4 as shown in FIG. 1.

### Synthesis of 5-Z/E-(2-naphthalen-2-yl-vinyl)-furo[2,3-d]pyrimidine-2,4-diamine (5)

To a solution of 5-(chloromethyl)furo[2,3-d]pyrimidine-2,4-diamine 4, (0.50 g, 2.5 mmol) in anhydrous DMSO (10 mL) tributylphosphine (0.85 g 3.75 mmol) was added, and the resulting mixture was stirred at 60° C. in an oil bath for 3 h under N₂ to form the phosphonium salt. The deep orange solution was then cooled to room temperature. To this solution was added sodium hydride (95% dispersion in mineral oil, 0.10 g, 3 mmol), followed by the 1-naphthalen-2-yl-ethanone (2.75 mmol). The reaction mixture was stirred at room temperature for 24-32 h. TLC showed the disappearance of the starting 5-(chloromethyl)furo[2,3-d]pyrimidine-2,4-diamine and the formation of two spots. The reaction was quenched with 20 mL methanol, washed with two portions of 50 mL methanol, and the resulting solution was evaporated under reduced pressure to dryness. To the residue was added 3 g of silica gel and CHCl₃ (20 mL) and the slurry was loaded onto a 4×20 cm dry silica gel column and flash chromatographed initially with CHCl₃ (300 mL), then sequentially with 2% MeOH in CHCl₃ (250 mL), 5% CH₃OH in CHCl₃ (300 mL). Fractions which showed the desired spot on TLC were pooled and evaporated to dryness and the residue was recrystallized from ethylacetate to afford a mixture of the E/Z isomers 5 (316 mg, 30%) as yellow needles: mp 238.2-247.5° C.; R_{f}=0.55 and 0.52 (CHCl₃/CH₃OH 5:1); ¹H-NMR (DMSO-d₆) (E/Z=2:1) E-isomer d 2.34 (s, 3H, 9-CH₃), 6.09 (s, 2H, 4-NH₂), 6.52 (s, 2H, 2-NH₂), 7.05 (s, 1H, 8-CH), 7.51-7.49 (m, 3H, 6-CH and C₁₀H₇), 7.96-7.89 (m, 4H, C₁₀H₇), 8.09 (s, 1H, C₁₀H₇); Z-isomer d 2.26 (s, 3H, 9-CH₃), 5.99 (s, 2H, 4-NH₂), 6.29 (s, 1H, 8-CH), 6.54 (s, 2H, 2-NH₂), 6.67 (s, 1H, 6-CH), 7.13-7.17 (m, 3H, C₁₀H₇), 7.70-7.89 (m, 4H, C₁₀H₇). Anal. (C₁₉H₁₆N₄O)C, H, N. This synthesis resulted in the production of 316 mg of E/Z isomers 5 as shown in FIG. 1.

### Synthesis of 5-E-(2-naphthalen-2-yl-vinyl)-furo[2,3-d]pyrimidine-2,4-diamine (1)

To a solution of 500 mg of E/Z mixture 5 in 60 mL of hot methanol 3 g of silica gel and CHCl₃ (20 mL) was added, and the slurry was loaded onto a 4×20 cm dry silica gel column and flash chromatographed initially with CHCl₃ (300 mL), then sequentially with 2% CH₃OH in CHCl₃ (500 mL), 5% CH₃OH in CHCl₃ (500 mL), and 8% CH₃OH in CHCl₃ (500 mL). Most fractions contained both the E and Z isomers. After three repetitions, chromatography of the collected mixture as mentioned above afforded fractions (75 mL) containing the pure product E-isomer (TLC). These were pooled and evaporated to afford analytically pure (20 mg) 1: mp 236-238° C.; ¹H-NMR(DMSO-d₆): δ 2.34 (s, 3H, 9-CH₃), 6.08 (s, 2H, 4-NH₂), 6.51 (s, 2H, 2-NH₂), 7.05 (s, 1H, 8-CH), 7.29-7.32 (d, 2H, —C₁₀H₇), 7.50-7.91 (m, 5H, C₁₀H₇), 8.09 (s, 1H, C₆—H). Analytical calculated for C₁₉H₁₆N₄O.0.16H₂O: C, 72.13; H, 5.10; N, 17.71. Found C, 71.16; H, 5.16; N, 17.36. This synthesis resulted in the production of 20 mg of E-isomer 1 as shown in FIG. 1.

### Example 2

### Reverse Phase HPLC Separation of E-Isomer 1 and Z-Isomer 6 from E/Z Mixture 5

Analytical Separation of E/Z Mixture 5

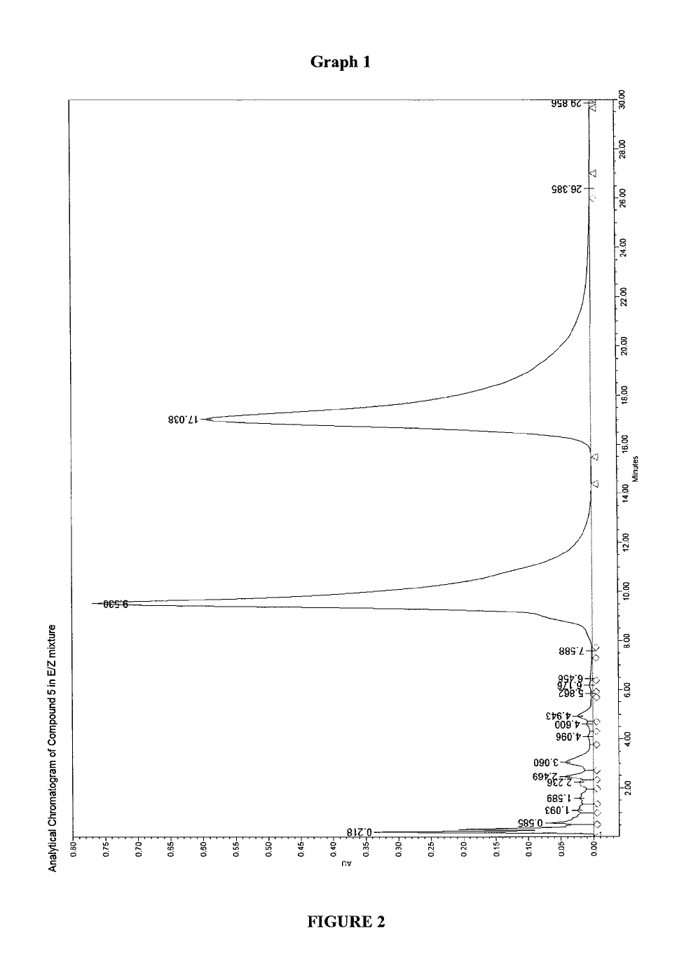

Reverse phase HPLC was utilized to separate the E-isomer 1 and Z-isomer 6 within the E/Z-mixture 5. The HPLC procedure is listed in Table 1 and the HPLC chromatogram is shown in FIG. 2. Previous NMR studies indicated that the peak with retention time 9.530 min corresponds to the Z-isomer 6, followed by the E-isomer 1 with retention time 17.038 min.

TABLE 1
Analytical HPLC Conditions

| | |
|---|---|
| Column | X-Bridge ® C-18; 4.6 × 50 mm |
| Detector | Waters ® 2487 Dual λ |
| | Absorbance Detector |
| Mobile Phase | A: water |
| | B: acetonitrile |
| Flow | 75% A and 25% B |
| | 2 ml/min |
| Sample | 1 solution in methanol |
| Injection Volume | 10 μL |
| Pump | Waters ® 600E |
| Retention Time (UV at 245 nm) | E-isomer 1: 17.038 min |
| | Z-isomer 6: 9.530 min |

Preparative Separation of E/Z-Mixture 5

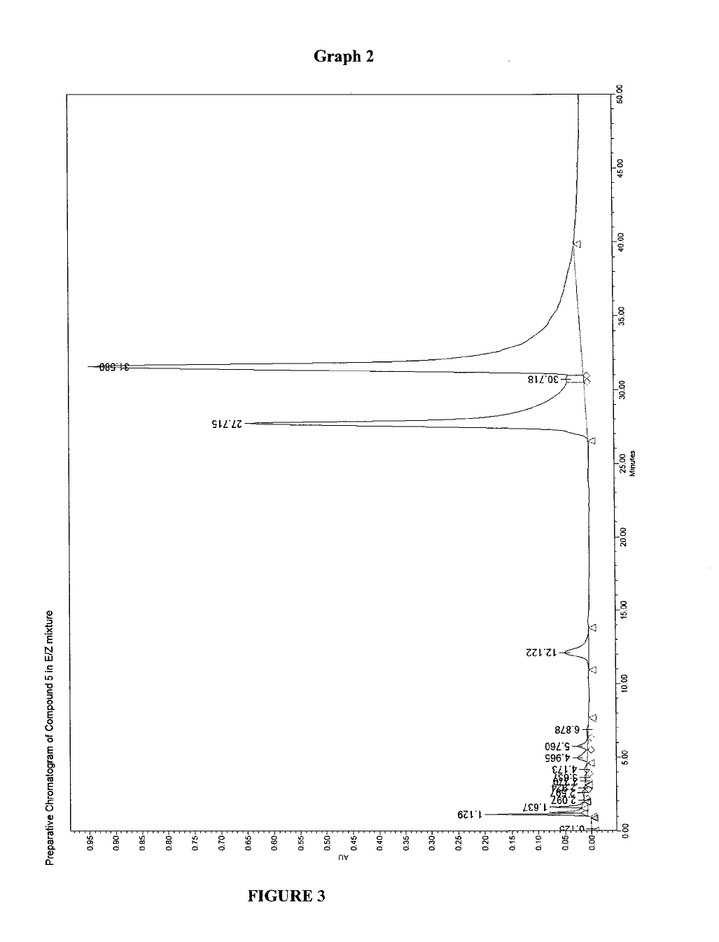

Reverse phase HPLC was utilized to separate the E-isomer 1 and Z-isomer 6 within the E/Z-mixture 5. The HPLC procedure is listed in the Table 2 and the HPLC chromatogram is shown in FIG. 3. Later NMR studies confirmed that the peak with retention time 22.715 min corresponds to the Z-isomer 6, followed by the E-isomer 1 with retention time 31.580 min.

TABLE 2
Preparative HPLC Conditions

| | |
|---|---|
| Column | X-Bridge ® C-18; 19 × 50 mm |
| Detector | Waters ® 2487 Dual λ |
| | Absorbance Detector |
| Mobile Phase | A: water |
| | B: acetonitrile |
| Flow | 0-1 min, 75% A and 25% B, 1 ml/min; |
| | 1-26 min, 75% A and 25% B, |
| | 30 ml/min; |
| | 26-50 min, 70% A and 30% B, |
| | 45 ml/min. |
| Sample | 1 in methanol |
| | (not necessarily clear solution) |
| Injection Volume | 1 ml, 10 mg contained |
| Pump | Waters ® 4000 |
| Fraction Collector | Waters ® Fraction Collector III |
| Retention Time (UV at 245 nm) | E-isomer 1: 31.580 min |
| | Z-isomer 6: 22.715 min |

### Example 3

### Biological Activity Difference of the E/Z Mixture 5 Compared with the Pure E-Isomer 1

Biological activity in angiogenic assays were chosen as a way to compare anti-angiogenic activity, potency an reversibility of inhibition. The studies were conducted on pieces of chick aorta placed in culture with a mixture of angiogenic agents and various compounds.

Unresolved mixture of E/Z-isomers 5 and isolated E-isomer 1 from the series of compounds under study were assessed for desirable biological activities, i.e., antiangiogenic activity and persistence of inhibitory activity. It is well known that binding vascular endothelial growth factor with antibodies or aptamers or inhibiting VEGF signaling with small molecules reduces and even reverses angiogenesis. However, angiogenesis rapidly resumes once the agent is removed (Mancuso et al., J. Clin. Invest., 116:610-21, 2006; Baffert et al., Am. J. Physiol. Heart, 290:H509-11, 2006). In the case of tumors, rapid regrowth of new vessels contributes to the regrowth and survival of the tumor. The testing of isolated E-isomer 1 revealed desirable, more persistent antiangiogenic activities compared with the E/Z mixture 5. In a model system, using the aortic ring assay (Auerbach, Clin. Chem., 49:32-40, 2003) to assess antiangiogenic agents, the E/Z mixture 5 produced 84% inhibition of capillary outgrowth, but when removed there was a resumption of angiogenesis. accounting for almost 72% of that observed in tissue not treated with antiangiogenic agents (control). In contrast, the E-isomer 1 isolated from the E/Z mixture 5 produced a comparable inhibition of angiogenesis but only a 31% regrowth of capillary-like structures on removal. This demonstrates improved persistence of activity, a beneficial biological activity of the isolated E-isomer 1 compared to the E/Z mixture 5.

These studies showed greater inhibition on a molar basis using the E-isomer than with the E/Z mixture of the parent compound. Additionally, these studies showed less resumption of angiogenesis with the E-isomer than with the E/Z parent compound. These properties are desirable for treating cancer where a persistent effect is desirable.

### Example 5

### Chemistry, Crystal Structure and Biology

Different reaction conditions for utilizing the following reaction conditions were optimized: (1) triphenylphosphine, potassium carbonate and protic solvents, such as MeOH and I-PrOH and others; (2) triphenylphosphine, sodium hydride and aprotic solvents, such as DMF and DMSO and others; and (3) phenyl lithium and tributylphosphine.

The X-ray crystal structure of the E-isomer of the 2-OMeC₆H₄-substituted analog of the 2,4 diaminofuro[2,3-d]pyrimidine with dihydrofolate reductase indicated that the molecule is flipped by a 180 degree rotation about the 2NH₂—C₂ bond compared with methotrexate (MTX). In this flipped binding, the 4-NH₂ group binds where the N₈ of MTX binds and the furo oxygen binds where the 4-NH₂ of MTX binds. Essentially, this demonstrated that the E-2-OMeC₆H₄ isomer binds as predicted in the flipped mode (Gangjee, A. Bioorg. Chem., 13:5475, 2005) and gives a physical basis for the E stereoisomer activity in biological assays.

What is claimed is:

1. A stereoselective method for preparing isolated E- and Z-isomers of 2,4-substituted-5-vinylfuro[2,3-d]pyrimidine and pharmaceutically acceptable salts, solvates and prodrugs thereof, comprising:
a. synthesizing said isolated E- and Z-isomers using at least one 2,4-substituted-5-(chloromethyl)furo[2,3-d]pyrimidine and at least one 2-substituted ketone using reaction conditions and reagents; and
b. separating said isolated E- and Z-isomers using at least one method selected from the group consisting of physical separation, chromatography and HPLC, wherein said isolated E- and Z-isomers each have the following composition: 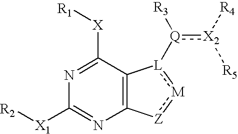
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkenyl, a heteroaroyl and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond, and
wherein said M is a carbon when said third chemical bond is a single bond.

2. The method according to claim 1, wherein said R₁ and said R₂ include the same or different substituents selected from the group consisting essentially of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

3. The method according to claim 1, wherein said R₃, said R₄ and said R₅ include the same or different substituents selected from the group consisting essentially of mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

4. The method according to claim 1, wherein either or both of said isolated E-isomers or said isolated Z-isomers inhibit at least one tyrosine kinase during angiogenesis.

5. The method according to claim 1, wherein either or both of said isolated E-isomers or said isolated Z-isomers inhibit a folate pathway required for cell growth.

6. The method according to claim 1, wherein either or both of said isolated E-isomers or said isolated Z-isomers are anti-angiogenic agents.

7. The method according to claim 1, wherein either or both of said isolated E-isomers or said isolated Z-isomers are anti-cancer agents.

8. A composition of 2,4-substituted-5-vinylfuro[2,3-d]pyrimidine and pharmaceutically acceptable salts, solvates, and prodrugs thereof comprising: 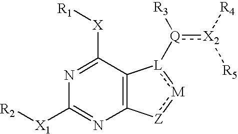
wherein X₁ and X₂ are independently selected from the group consisting of an alkyl group, an alkenyl group, a heteroalkyl group, a heteroalkenyl group, a heteroaroyl group and a heteroatom,
wherein R₁ and R₂ are selected from the same or different group consisting of a hydrogen, an alkyl, an alkenyl, an aryl, an aroyl, a heteroalkyl, a heteroalkeyl, and a heteroallyl,
wherein R₃, R₄, and R₅ are selected from the same or different group consisting of a hydrogen, an alkyl, an alkenyl, an aryl, an aroyl, a heteroalkyl, a hetereoalkeyl, and a heteroallyl,
wherein Z is selected from the group consisting of C, CH, CH₂, N, NH, S, and O,
wherein L is selected from the group consisting of C, CH, CH₂, N, NH, CH═CH, CH═N, and N═CH,
wherein a first chemical bond between said L and said M is selected from the group consisting of a single bond and a double bond,
wherein M consists of CH and said first chemical bond is a single bond or wherein M consists of C and said first chemical bond is a double bond,
wherein Q is selected from the group consisting of C, CH, and CH₂,
wherein a second chemical bond between said Q and said X₂ is selected from the group consisting of a single bond and a double bond,
wherein said second chemical bond between said Q and said X₂ is a double bond when said R₃ is a hydrogen or an alkyl group,
wherein a third chemical bond between said M and said Z is selected from the group consisting of a single bond and a double bond,
wherein said M is a carbon when said third chemical bond is a single bond, and
wherein said composition contains a mixture of E- and Z-isomers that are isolatable.

9. The composition of claim 8, wherein said R₁ and said R₂ include the same or different substituents selected from the group consisting essentially of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

10. The composition of claim 8, wherein said R₃, said R₄ and said R₅ include the same or different substituents selected from the group consisting essential of a mono-substituted aryl group, a di-substituted aryl group, a tri-substituted aryl group, an unsubstituted aryl group, a straight lower alkyl group with 1 to 6 backbone carbons, a branched lower alkyl group with 1 to 6 backbone carbons, and a cyclic lower alkyl group with 1 to 6 backbone carbons.

11. The composition of claim 8, wherein E-isomers and Z-isomers are isolated from said composition, and wherein said isolated E-isomers and isolated Z-isomers each have both cytostatic and cytotoxic activity.

12. The composition of claim 11, wherein either or both of said isolated E-isomers or isolated Z-isomers of said composition inhibit growth factors, said growth factors selected from the group consisting of VEGF and PDGF, and wherein the receptor for said VEGF is involved in the initial phases of angiogenesis and the receptor for said PDGF is involved in the stabilization of new capillaries.

13. The composition of claim 11, wherein either or both of said isolated E-isomers and isolated Z-isomers inhibit a folate pathway required for cell proliferation.

14. A method of treating at least one disease in a patient, comprising administering to said patient in a pharmaceutically effective amount one or more isolated isomers of the composition as claimed in claim 8.

15. The method according to claim 14, wherein said isolated isomers are E-isomers and Z-isomers.

16. The method according to claim 15, wherein said isolated E-isomer is administered to said patient.

17. The method according to claim 15, wherein said isolated Z-isomer is administered to said patient.

18. The method according to claim 15, wherein said isolated E-isomer is administered at one time point to said patient to treat at least one disease in said patient and said isolated Z-isomer is administered at a different time to said patient to treat the same or different at least one disease in said patient.

19. The method according to claim 15, wherein said isolated E-isomer and said isolated Z-isomer is administered simultaneously to said patient to treat at least one disease in said patient.

20. The method according to claim 14, wherein said at least one disease is selected from the group consisting of rheumatoid arthritis, wet form of macular degeneration and cancer.

21. The method according to claim 14, wherein either or both of said isolated E-isomer or said isolated Z-isomer inhibits at least one tyrosine kinase during angiogenesis.

22. The method according to claim 14, wherein either or both of said isolated E-isomer or said isolated Z-isomer inhibits a folate pathway required for cell growth.

23. The method according to claim 14, wherein either or both of said isolated E-isomer or said isolated Z-isomer is an anti-angiogenic agent.

24. The method according to claim 14, wherein either or both of said isolated E-isomer and said isolated Z-isomer is an anti-cancer agent.

25. A method to reduce aberrant angiogenesis in a patient afflicted with aberrant angiogenesis, comprising administering in a pharmaceutically effective amount either or both isolated E-isomer or isolated Z-isomer of the composition as claimed in claim 8 to said patient, wherein said either or both isolated E-isomer or isolated Z-isomer is administered simultaneously or at different times to said patient.

26. The method according to claim 24, wherein said aberrant angiogenesis is reduced in diseases selected from the group consisting of rheumatoid arthritis, wet form of macular degeneration and cancer.
